(19) United States
(12) Patent Application Publication
(10) Pub. No.: US 2005/0256483 A1
(43) Pub. Date: Nov. 17, 2005

(54) Disposable vaginal insertion device

(76) Inventors: Ann Marie Przepasniak, Appleton, WI (US); MaryAnn Zunker, Oshkosh, WI (US); Cornelis J. Bosselaar, Appleton, WI (US)

Correspondence Address: SCHWEGMAN, LUNDBERG, WOESSNER & KLUTH, P.A., P.O. BOX 2938, MINNEAPOLIS, MN, 55402-0938, US

(21) Appl. No.: 10/837,537

(22) Filed: Apr. 30, 2004

(51) Int. Cl. A61M 35/00; A61M 37/00; A61F 13/15; A61F 13/20; A61B 10/00

(52) U.S. Cl. 604/385.17; 604/385.18; 604/1

(57) ABSTRACT

The invention relates to a disposable vaginal insertion device. The disposable device includes a housing having a first end and a second end and a core that is positioned within the housing. The core includes a handle and a swab that is secured to the handle. The core is axially movable within the housing between a first position where the swab is at least partially within the housing and a stop on the handle is outside the housing, and a second position where the swab is extended through the second end of the housing until the stop on the handle engages the housing to prevent further insertion of the handle into the housing. In another embodiment, the disposable device includes a core that is capable of rotating within the housing such that the handle of the core periodically engages the housing to indicate a relative amount of core rotation.

### FIELD OF THE INVENTION

Some embodiments of the present invention relate to a vaginal insertion device, and in particular to a disposable vaginal insertion device.

### BACKGROUND OF THE INVENTION

A typical vagina is a relatively long and tube-like structure that extends from the cervix (i.e., outer end of the uterus) down to the labia minora. A vagina will normally emit secretions from glands in the vaginal lining and the cervical canal of the uterus. As an example, mucous is developed in the cervical canal of the uterus during the phase of the menstrual cycle where the endometrium thickens and glands in the endometrium secrete mucous.

Vaginal secretions are typically clean but occasionally debris in the form of blood or seminal fluid can accumulate within the vagina. When debris accumulates within the vagina it may be beneficial to treat the vagina with fragrances, medications, germicides and/or deodorants (among other items).

One method of treating the vagina includes using douching products. Some conventional douching products include a nozzle that is attached to a bottle filled with a cleaning solution. The cleaning solution is forced through the nozzle into the vagina under pressure. One drawback with such douching products is that bacteria can be forced up into the cervix when too much pressure is applied to force the cleaning solution up into the vagina. In addition, these types of products can rinse out healthy, normal flora (lactobacillus) along with unwanted materials, such as semen, discharges and bacteria.

Other conventional douching products are applied to the vagina as films that are later discharged from the body. One drawback with film-based douching products is that they are difficult to insert up into the vagina. Another drawback is that the film is discharged from the vagina over time such that a pantiliner is often required for a couple of days after the film is applied to prevent staining in undergarments.

There are some vaginal cleaning products that are designed to provide mechanical cleaning of the vagina without using free flowing fluid. These types of devices are typically inserted into the vagina and mechanically manipulated in order in clean the vagina.

One drawback with using such devices is that they are difficult to properly insert into the vagina because it is difficult to determine how far the device is being inserted into the vagina. Another drawback with using such devices is that it is difficult to tell just how much the device is being manipulated within the vagina because most of the device is hidden from view within the vagina during cleaning.

There is a need for a vaginal insertion device that is able to mechanically clean inside a vagina and/or apply a material inside the vagina. The vaginal insertion device should be able to be inserted into the vagina without irritating the vagina. The vaginal insertion device should also provide one indicator as to how far the device is inserted into the vagina and/or another indicator as to how much the insertion device is being rotated within the vagina.

### SUMMARY OF THE INVENTION

In response to the drawbacks described above with respect to conventional vaginal douching products and cleaning devices, a disposable vaginal insertion device has been invented.

One aspect of the invention relates to a disposable vaginal insertion device. The disposable device includes a housing having a first end and a second end and a core that is positioned within the housing. The core includes a handle and a swab that is secured to the handle. The core is axially movable within the housing between a first position where the swab is at least partially within the housing and a stop on the handle is outside the housing, and a second position where the swab is extended through the second end of the housing and the stop on the handle engages the housing to prevent further insertion of the handle into the housing. In some embodiments, the swab is positioned entirely within the housing when the core is in the first position and is positioned entirely outside the housing when the core is in the second position. The core may also be able to rotate within the housing such that the handle of the core periodically engages the housing to provide an indication as to how far the core is rotating within the housing.

Another aspect of the invention relates to another disposable vaginal insertion device. The disposable device includes a housing having a first end and a second end, and a core that is positioned within the housing. The core includes a handle and a swab that is secured to the handle. The core is able to rotate within the housing such that the handle of the core periodically engages the housing to provide an indication as to how far the core is rotating within the housing. In some embodiments, the core and the housing emit a sound when the handle periodically engages the housing. The handle may engage the housing one or more times per revolution of the core relative to the housing. In some embodiments, the core may be axially movable between a first position where the swab is at least partially within the housing and a stop on the handle is outside the housing, and a second position where the swab is extended through the second end of the housing and the stop on the handle engages the housing to prevent further insertion of the handle into the housing.

Another aspect of the invention relates to a method of engaging a vagina. The method includes inserting a disposable vaginal insertion device into a vagina. The disposable device includes a housing having a first end and a second end and a core that is positioned within the housing. The core includes a handle and a swab that is secured to the handle. The core is inserted into the vagina when the core is in a first position where the swab is at least partially within the housing and a stop on the handle is outside the housing. The method further includes axially moving the core to a second position where the swab is extended through a second end of the housing further into the vagina until the stop on the handle engages the housing to prevent further insertion of the swab into the vagina. In some embodiments, the method may further include rotating the handle of the core to clean the vagina with the swab. Rotating the handle to clean the vagina with the swab may include engaging the handle with the housing to provide an indication as to how far the core is rotating within the housing.

Still another aspect of the invention relates to another method of engaging a vagina. The method includes inserting a disposable vaginal insertion device into a vagina. The disposable device includes a housing having a first end and a second end and a core that is positioned within the housing. The core includes a handle and a swab that is secured to the handle. The method further includes rotating the handle relative to the housing to clean the vagina with the swab and to periodically engage the handle with the housing. Periodically engaging the handle with the housing provides an indication as to how far the core is rotating within the housing. In some embodiments, inserting the disposable device into the vagina may include inserting the disposable device into the vagina when the core is in a first position where the swab is at least partially within the housing and a stop on the handle is outside the housing, and then axially moving the core to a second position where the swab is extended from the second end of the housing further into the vagina until the stop on the handle engages the housing to prevent further insertion of the swab into the vagina. In addition, rotating the handle of the core to periodically engage the handle with the housing may include generating a sound by periodically engaging the handle with the housing, and/or periodically applying a force that overcomes resistance generated by engaging the handle with the housing.

### BRIEF DESCRIPTION OF THE DRAWINGS

The above-mentioned and other features will become more apparent and better understood by reference to the following description of the invention taken in conjunction with the accompanying drawings.

FIG. 1 is a side view illustrating an example disposable vaginal insertion device with a core of the device in a first position.

FIG. 2 is a section view of the disposable vaginal insertion device shown in FIG. I taken along line 2-2 in FIG. 1.

FIG. 3 is a side view illustrating the disposable vaginal insertion device shown in FIG. 1 with the core of the device in a second position.

FIG. 4 is a section view of the disposable vaginal insertion device shown in FIG. 3 taken along line 4-4 in FIG. 3.

FIG. 5 is a section view of the disposable vaginal insertion device shown in FIG. 3 taken along line 5-5 in FIG. 3.

FIG. 6 is a section view similar to FIG. 5 illustrating the core rotated from the position shown in FIG. 5.

FIG. 7 is a section view similar to FIG. 5 illustrating a cross-section of another example disposable vaginal insertion device.

FIG. 8 is a section view similar to FIG. 5 illustrating a cross-section of another example disposable vaginal insertion device.

FIG. 9 is a side view illustrating another example disposable vaginal insertion device with a core of the device in a first position.

FIG. 10 is a section view of the disposable vaginal insertion device shown in FIG. 9 taken along line 10-10 in FIG. 9.

FIG. 11 is a side view of the disposable vaginal insertion device shown in FIGS. 9 and 10 with the core of the device in a second position.

FIG. 12 is a section view of the disposable vaginal insertion device shown in FIG. 11 taken along line 12-12 in FIG. 11.

FIG. 13 is a section view of the disposable vaginal insertion device shown in FIG. 9 taken along line 13-13 in FIG. 9.

FIG. 14 is a section view similar to FIG. 13 illustrating a cross-section of another example disposable vaginal insertion device.

FIG. 15 is a section view similar to FIG. 13 illustrating a cross-section of another example disposable vaginal insertion device.

FIGS. 16-17 are schematic views illustrating an example method of engaging a vagina.

FIGS. 18-19 are schematic views illustrating another example method of engaging a vagina.

### DESCRIPTION OF THE INVENTION

The disposable vaginal insertion device of the present invention may provide an efficient, healthy and low-cost way to clean inside a vagina without using a douching product. The disposable device may also provide an effective way to apply a material inside a vagina.

As used herein, “disposable device” includes a device that is disposed of after use and is not intended for reuse. The disposable device described herein may be especially useful to an individual that needs to mechanically clean (or apply materials to) their vagina in a variety of situations.

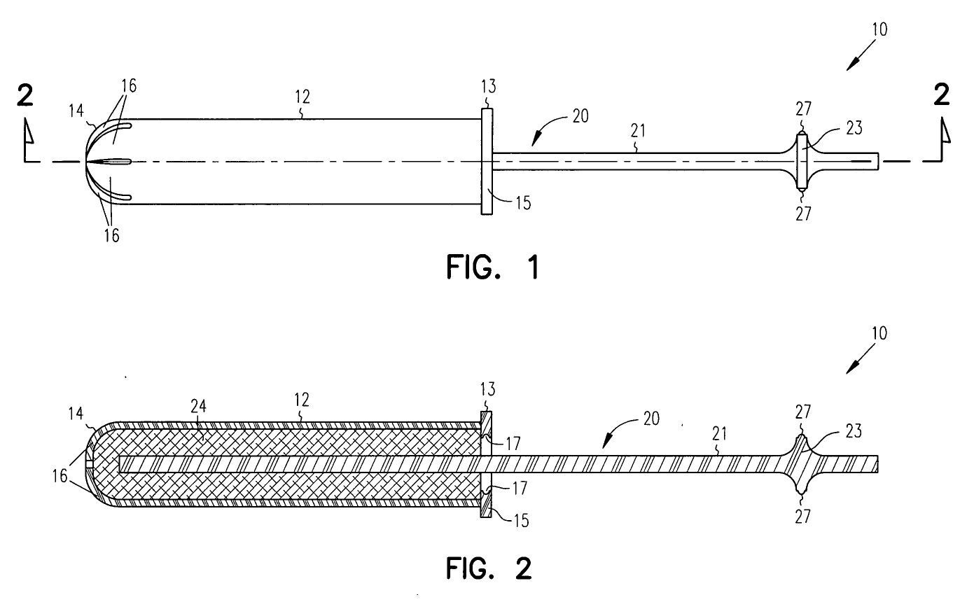

FIGS. 1-4 illustrate an example disposable vaginal insertion device 10 of the present invention. The disposable device 10 includes a housing 12 having a first end 13 and a second end 14. The disposable device 10 further includes a core 20 that is within the housing 12. The core 20 includes a handle 21 and a swab 24 that is secured to the handle 21. In the illustrated example embodiment, the handle 21 is embedded in the swab 24.

As used herein, the term “securing” means permanently bonding. Any securing, or bonding, of the handle 21 to the swab 24 may occur at any time during the fabrication of the disposable device 10. Some of the example methods that may be used to bond the swab 24 to the handle 21 include adhesive bonding, thermal bonding and ultrasonic bonding (among others).

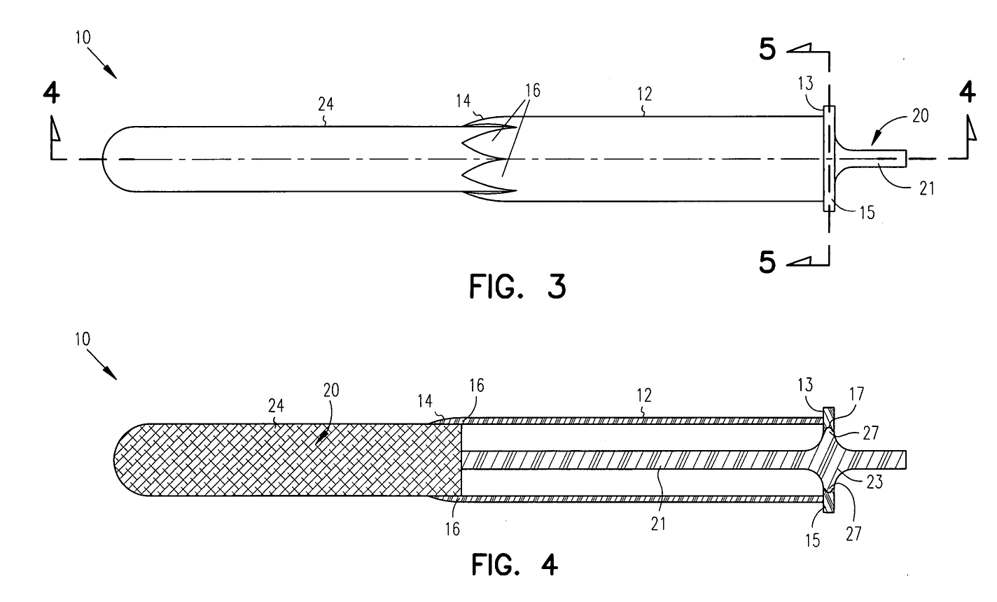

The core 20 is axially movable within the housing 12 between a first position (FIGS. 1 and 2) and a second position (FIGS. 3 and 4). In the first position, the swab 24 may be at least partially within the housing 12 while a stop 23 on the handle 21 is located outside the housing 12. In the second position, the swab 24 is extended through the second end 14 of the housing 12 until the stop 23 on the handle 21 engages the housing 12 to prevent further insertion of the handle 21 into the housing 12. The swab 24 may be positioned entirely within the housing 12 when the core 20 is in the first position and/or may be positioned entirely outside the housing 12 when the core 20 is in the second position. In some embodiments, the swab 24 has a length that may be between 50 mm and 150 mm and a width that may be between 25 mm and 50 mm.

The swab 24 may be constructed of any material that is suitable for insertion into a vagina. Some example materials include cotton, rayon, wood pulp and polymeric substances such as nonwoven fabrics, foam sponges and thermoplastics.

The swab 24 may be designed with any type of surface that enhances the disposable device's cleaning properties. In some embodiments, the surface of the swab 24 may be textured to facilitate removal of residue and film from the vagina.

In some embodiments, the swab 24 may be composed of a nonwoven fabric. As used herein, “nonwoven fabric” refers to a web or fabric (made partly or wholly of non-cellulose material) that includes individual fibers which are interlaid (randomly or in a pattern) in a knitted fabric. The nonwoven webs may be formed many processes, including meltblowing, spunbonding and bonded carded. The nonwoven fabrics that are used in the swab 24 may be produced from polymers such as polyethylene or polypropylene.

The swab 24 may also be formed of a nonwoven fabric that is made of an array of interbonded thermoplastic fibers. The fibers may be formed from a wide variety of thermoplastic materials. Some example thermoplastic materials include polyolefins (e.g., polyethylene, polypropylene), polystyrene and polyamides (e.g., nylon). In addition, thermoplastic polymers that are elastomeric may also be used as fibers. Some example elastomeric materials include polyurethanes and block copolymers. It should be noted that blends of any of the foregoing materials may be used to form the fibers.

The fibers may also include additives (e.g., wax, pigments, stabilizers and fillers) that are inserted as the fibers are fabricated to achieve one or more desired properties within the fibers. Some example additives include compatible surfactants that are added to the polymers to make the surface of the fibers more wettable and thereby improve the ability of the swab 24 to attract unwanted debris within the vagina. It should be noted that the amount of surfactant that is added to the fibers can be adjusted to control the surface wetting of the fabric that is formed from the fibers. Some example surfactants that may be added to the fibers include sodium dioctyl sulfosuccinate and alkyl phenoxy ethanol (among others).

In some embodiments, the swab 24 may include a cover material (not shown) that is placed on the swab 24 to prevent fiber sloughing. In addition, some cover materials may improve the wettablilty of the swab 24. There also cover materials that may provide stain-masking. In addition, some cover materials may be capable of capturing and/or storing substances within the cover material itself, or trapping the substances within the inner portion of the swab 24.

As examples, the cover material may store and/or capture debris, cleansers, lubricants, spermicidal agents and medications (among other materials) before, or after, using the disposable device 10. Some example cover materials include spunbound, spunlace, bonded carded web and apertured film materials. In one example embodiment, the cover material is an apertured film that is formed of a polyolefin which may or may not be combined with a nonwoven fabric.

When a nonwoven fabric is used as the material for the swab 20, the basis weight of the nonwoven fabric may vary depending on the properties that are desired within the swab 20. As an example the basis weight for the nonwoven fabric may be as low as 10 grams per square meter and as high as 300 grams per square meter.

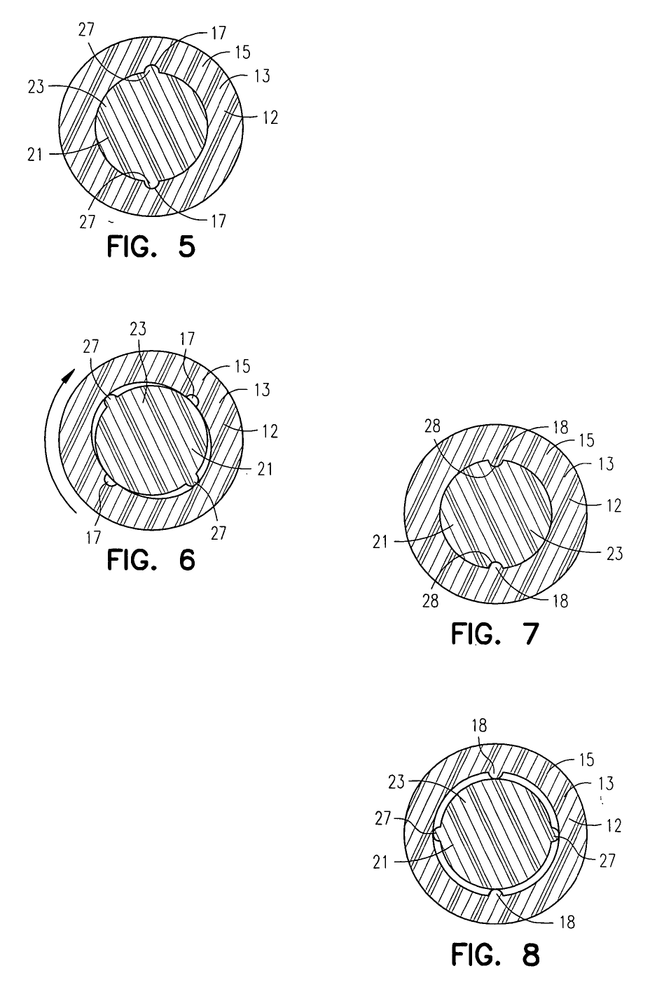

In the example embodiment illustrated in FIGS. 1-4, the stop 23 is an annular disk that projects from the handle 21 to engage the first end 13 of the housing 12 when the core 20 is in the second position (FIGS. 3 and 4). As shown most clearly in FIG. 5, the housing 12 includes a projection (e.g., end cap 15) that extends into an opening in the housing 12 to engage the stop 23 on the handle 21 when the core 20 is in the second position. It should be noted that in other embodiments the housing 12 may not include an end cap such that a larger annular disk directly engages the wall of the housing 12 at the first end 13 of the housing 12.

The housing 12 helps to protect the swab 24 from contamination when the core 20 is in the first position. In the example embodiments illustrated in FIGS. 1-4, the housing 12 is a cylindrical tube. It should be noted that the cross-section of the housing 12 may take a variety of forms (e.g., circle, square, rectangle and polygon) depending on the properties that are desired within the disposable device 10.

The housing 12 may be made from a material that is non-toxic, low cost and hygienic. Some example materials that may be used for housing 12 include polyethylene and polypropylene (among other materials). In some embodiments, the housing 12 may be made from a combination of materials.

The width (i.e., outer diameter) of the housing 12 may vary depending on the functional properties that are desired of the disposable device 10. In some embodiments, the housing may be small enough to make it easy for a user to insert the disposable device 10 into the vagina while being wide enough to give the housing 12 sufficient space to store a large enough swab 24 for cleaning inside the vagina and/or applying a material to the vagina. As an example, the width of the housing 12 may be between 25 mm and 60 mm.

The wall thickness of the housing 12 should be large enough to provide sufficient rigidity to the disposable device 10 during vaginal insertion. As an example the wall thickness may be between 2 mm and 10 mm. In addition, the length of he housing 12 may be between 60 mm and 150 mm. The relative lengths of the housing 12 and the swab 24 may vary depending on the desired properties of the disposable device 10.

In some embodiments, the second end 14 of the housing 12 includes resilient members 16 that at least partially close the second end 14 of the housing 12. As the core 20 is moved from the first position to the second position, the resilient members 16 are forced apart by the swab 24 (see FIG. 3). Although the resilient members 16 are shown as relatively thin and somewhat wedge-shaped sections, the resilient members 16 may be a variety of shapes and sizes.

The core 20 may also be able to rotate within the housing 12 such that the handle 21 periodically engages the housing 12 as the core 20 rotates within the housing 12. When the handle 21 periodically engages the housing 12 during rotation, a user of the disposable device 10 is provided with an indication as to how far the core 20 is rotating within the housing 12.

In the example illustrated embodiment, the handle 21 engages the housing 12 during rotation when the core 20 is in the second position. As shown in FIG. 5, the stop 23 includes members 27 that are on opposing sides of the stop 23. The members 27 are received in detents 17 that are on opposing sides of the end cap 15.

FIG. 6 shows the members 27 as the handle 21 is rotated to disengage the members 27 from the detents 17 in the housing 12. As the core 20 continues to rotate, the members 27 on the stop 23 eventually reengage the detents 17 in the end cap 15 to provide an indication to the user that the core 20 has rotated at least one-half of a revolution. During insertion a user will be able to hear and/or feel when the members 27 engage the detents 17 such that disposable device 10 indicates to the user that the handle 21 has been rotated one-half of a revolution relative to the housing 12.

Although the stop 23 is shown with two members 27 and the housing 12 is shown with two detents 17, it should be noted that any number of members 27 and detents 17 may be included on the stop 23 and the housing 12. As an example, the stop 23 may include one section and the housing 12 may include one detent such each time the member engages the detent the core 20 has been rotated one full revolution.

In the example embodiment illustrated in FIG. 7, there may be detents 28 on the stop 23 (or another portion of handle 21) while the housing 12 includes projections 18 that engage the detents 28 on the handle 21. It should be noted that any number and/or combination of members, projections and detents may be included on handle 21 and housing 12.

FIG. 8 shows an example embodiment where the handle 21 includes members 27 that extend from the handle 21 and the end cap 15 includes projections 18 that extend into the opening in the housing 12. As the handle 21 rotates within the housing 12, the members 27 and the projections 18 engage one another to provide an indication to a user as to how far the core 20 is rotating within the housing 12.

The handle 21 may be a variety shapes and sizes. In addition, the cross-section of the handle 21 may take a variety of forms (e.g., circle, square, rectangle and polygon) depending on the properties that are desired within the disposable device 10.

The handle 21 may be made from any material that is suitable for use in a disposable vaginal insertion device 10. The handle 21 should be made from a material that is non-toxic, low cost and hygienic. Some example materials that may be used for handle 21 include paper, cardboard, polyethylene and polypropylene (among other materials). In some embodiments, the handle 21 may be made from a combination of materials.

It should be noted that handle 21 may be solid or hollow. When the handle 21 is hollow, the wall thickness of the handle 21 should be large enough to provide sufficient rigidity to the disposable device 10.

In some embodiments, the handle 21 may have a length that is between 40 mm and 250 mm. The relative lengths of the swab 24, housing 12 and handle 21 may vary depending on the desired properties of the disposable device 10. The handle 21 and the swab 24 may be sized to keep the handle 21 far enough outside the vagina when the core 20 is in the second position to allow a user to adequately manipulate the swab 24 via the handle 21. In addition, the handle 21 and the swab 20 may be sized to be short enough to allow a user to generate sufficient pressure on the vagina with the swab 24 via the handle 21.

The width (i.e., outer diameter) of the handle 21 may be small enough to make it easy for a user to insert the disposable device into the vagina while being wide enough to give the handle 21 sufficient rigidity to properly clean inside the vagina and/or apply a material to the vagina. As an example, the width of the handle 21 may be between 3 mm and 50 mm depending in part on the material that used for the handle 21.

Many other embodiments are contemplated for the handle and/or the stop. FIGS. 9-12 illustrate another example disposable vaginal insertion device 50 of the present invention. The disposable device 50 includes a housing 52 having a first end 53 and a second end 54. The disposable device 50 further includes a core 60 that is within the housing 52. The core 60 includes a handle 61 and a swab 64 that is secured to the handle 61. In the illustrated example embodiment, the swab 64 is embedded in the handle 61, although in other embodiments the handle 61 may be embedded in the swab 64 depending on the relative sizes of the handle 61 and the swab 64.

The swab 64 may be similar to the swab 24 shown in FIGS. 1-4. In addition, the housing 52 may be similar to the housing 12 shown in FIGS. 1-4. The housing 52 includes resilient members 66 that are similar to the resilient members 16 shown in FIGS. 1-4. However, there is no end cap on the first end 53 of the housing 52.

The core 60 may be able to rotate within the housing 52 such that the handle 61 of the core 60 periodically engages the housing 52 as the core 60 rotates within the housing 52. The periodic engagement between the handle 61 and the housing 52 indicates to a user how far the core 60 is rotating within the housing 52.

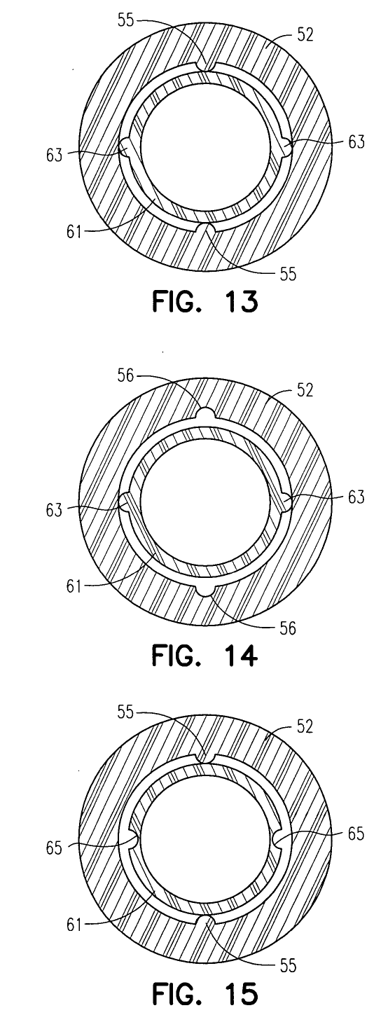

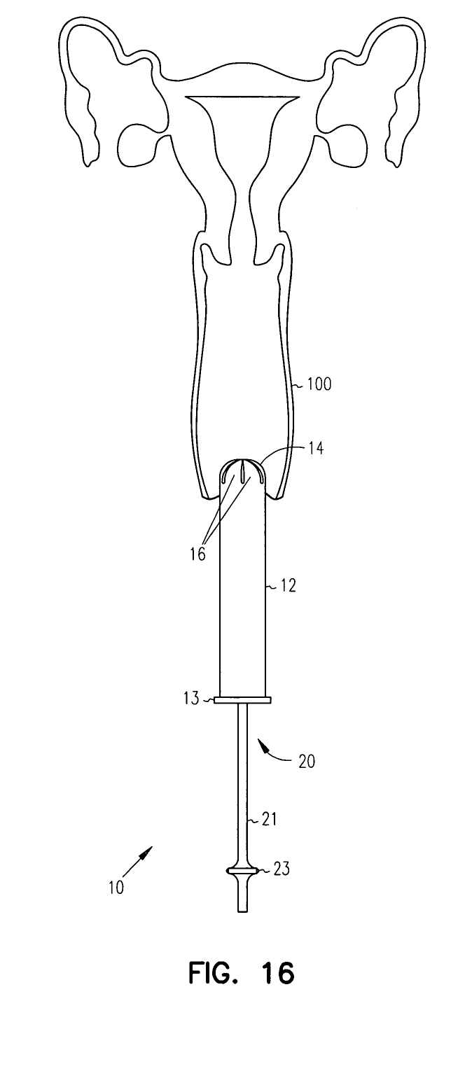

The handle 61 on the core 60 may engage the housing 52 in any number of configurations. FIGS. 9 and 13 show that handle 61 may include one or more members 63 and housing 52 may include one or more projections 55 which periodically engage the members 63 as the core 60 rotates within the housing 52. FIG. 14 shows an alternative embodiment where the housing 52 may include one or more detents 56 that periodically receive the members 63 on the handle 61 as the core 60 rotates within the housing 52. In addition, FIG. 15 shows an alternative embodiment where the housing 52 may include one or more projections 55 and the handle 61 may include one or more detents 65 that periodically receive the projections 55 as the core 60 rotates relative to the housing 52.

In some embodiments, the core 60 and the housing 52 emit a sound (e.g., a click) when the handle 61 periodically engages the housing 52 during rotation of the handle 61 within the housing 52. It should be noted that although the members 63 are shown in FIG. 9 as extending along the entire length of the handle 61, one or more of the members 63 may extend along only a portion of the handle 61. In addition, any number and/or combination of members, projections and detents may be included in the handle 61 and/or the housing 52.

As the core 60 rotates, the members 63 and/or detents 65 on the handle 61 engage the projections 55 and/or detents 56 on the housing 52 to provide an indication to the user that the core 20 has rotated at least one-half of a revolution relative to the housing 52. One-half of a revolution is indicated because the members and/or detents are on opposing sides of the handle 61 and the housing 52. In other embodiments, the handle 61 may include one member or one detent and the housing 52 may include one projection or one detent such each time the handle 61 engages the housing 52, the core 60 has been rotated one full revolution within the housing 52.

In the example embodiment illustrated in FIGS. 9-12, the handle 61 is a cylindrical tube. As an example, the width of the handle 61 may be between 3 mm and 50 mm, the length of the handle 61 may be between 40 mm and 250 mm, and thickness of the cylindrical tube wall may be between 2 mm and 10 mm.

The relative lengths and widths of the swab 64, housing 52 and handle 61 may vary depending on the desired properties of the disposable device. The cylindrical tube should have a wall thickness that is large enough to provide sufficient rigidity to the disposable device 50 during vaginal insertion. In addition, the materials and mechanical properties for the handle 61 may be similar in many respects to the materials and mechanical properties that were described above with regard to FIGS. 1-4 for handle 21.

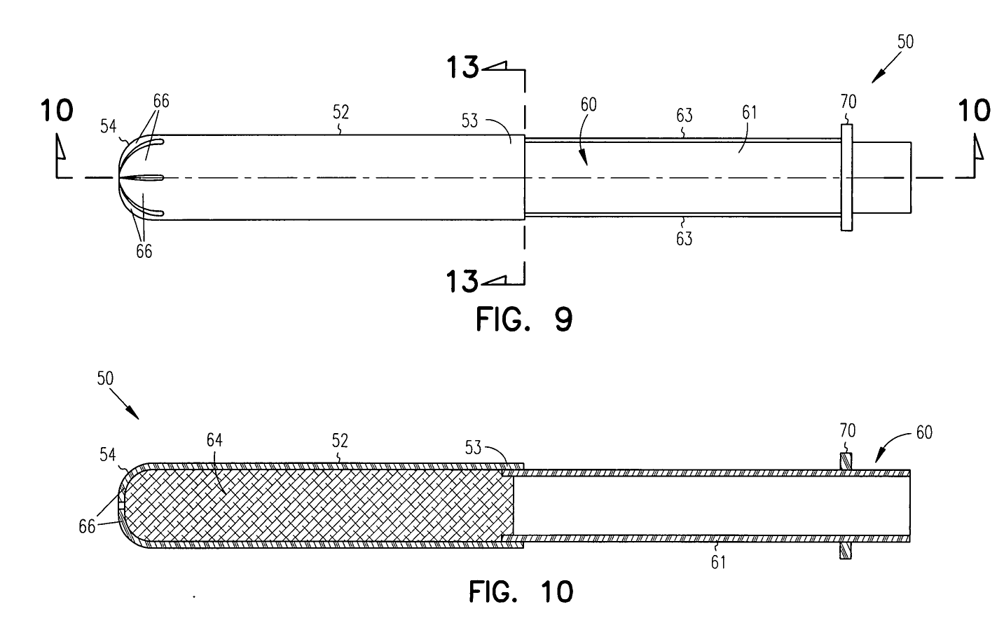

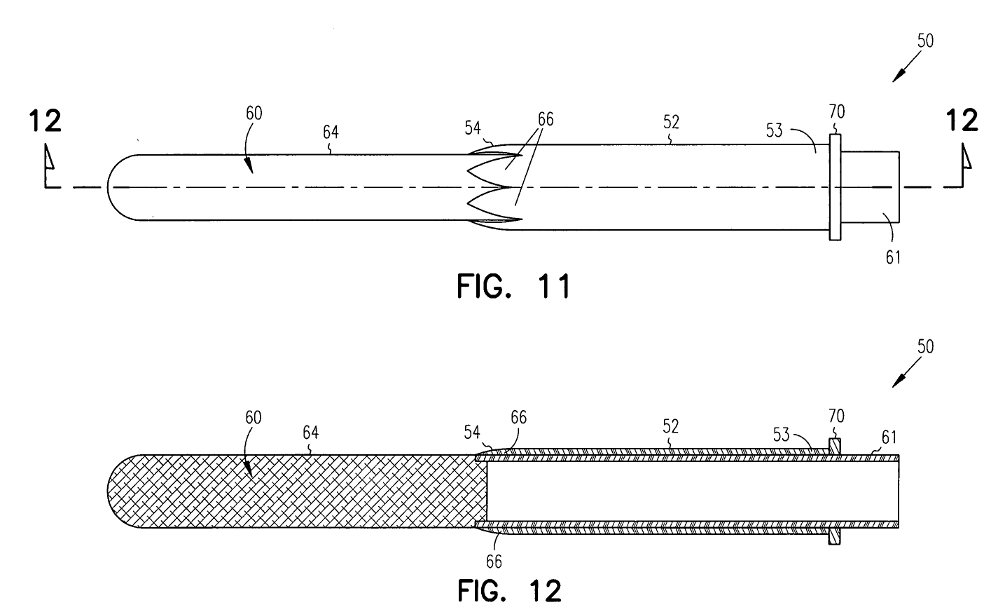

In some embodiments, the core 60 is axially movable within the housing 52 between a first position (FIGS. 10 and 11) and a second position (FIGS. 12 and 13). In the first position, the swab 64 is at least partially within the housing 52 and a stop 70 that is on the handle 61 is located outside the housing 52. In the second position, the swab 64 is extended through the second end 54 of the housing 52 until the stop 70 on the handle 61 engages the housing 52 to prevent further axial movement of handle 61.

In the example embodiment illustrated in FIGS. 9-12, the stop 70 on the handle 61 is an annular disk. As an example, the stop 70 is shown in FIGS. 11 and 12 as engaging the first end 53 of the housing 52. In some embodiments, the swab 64 may be positioned entirely within the housing 52 when the core 60 is in the first position and/or may be positioned entirely outside the housing 52 when the core 60 is in the second position.

In any of the disposable devices described or reference herein, a cleanser (not shown) may be applied to the swab of the disposable device. The term “cleanser” refers to any paste, gel, powder, oil, liquid or any other appropriate medium that may be used in vaginal cleansing. Some example cleansers include surfactants such as water-soluble polymers, polysorbates, glycerins, glycol-based surfactants and/or silicone-based surfactants. It should be noted that the swab may also include other materials, such as water, salts, vinegars, humectants, scouring powders, thickening agents and aromas (among others).

The cleanser may be applied to the swab 20 such that the cleanser is moist to the touch, or the cleanser may be dry and then activated upon contact with the vaginal tissue. The cleanser should not kill significant amounts of lactobacillus while removing unwanted debris during mechanically cleaning of the vagina. In addition, the cleanser should not contain ingredients that might lead to dry vaginal mucosal tissue.

In some embodiments, the cleanser may include a moisturizer that helps to maintain a normal hydration level after mechanical cleaning. The cleanser may also contain preservatives and other ingredients that do not disrupt the normal flora of the vaginal vault (e.g., sorbic acid, citric acid, methyl paraben and natural preservatives such as grapefruit extract).

The cleanser may be applied to the elongated swab 20 by various means including spray-coating, dipping, doning, impregnating, electro-spinning coating and inkjet printing (among others). In addition, the cleanser may be applied to the elongated swab 20 at any point in time. As examples, the cleanser may be applied to the elongated swab 20 just prior to using the disposable device 10 to mechanically clean a vagina, or the cleanser may be “pre-applied” to the elongated swab 20. As used herein, “pre-applied” refers to applying the cleanser to the elongated swab 20 during a manufacturing process of the disposable device 10 and/or the elongated swab 20. In addition, “pre-applied” also refers to applying the cleanser to the elongated swab 20 before the sale of disposable device 10.

It should be noted that other materials besides cleansers may be applied to the elongated swab 20 such that the disposable device 10 can be used to apply materials to the vagina. Although any appropriate material may be added to the elongated swab 20, some example materials include lubricants and other inactive or active ingredients (e.g., a spermicidal agent or a medication). Another example material may be a motility enabler that facilitates sperm motility. Any of the materials that may be applied to the elongated swab 20 may be in any form (e.g., liquid, powder and paste).

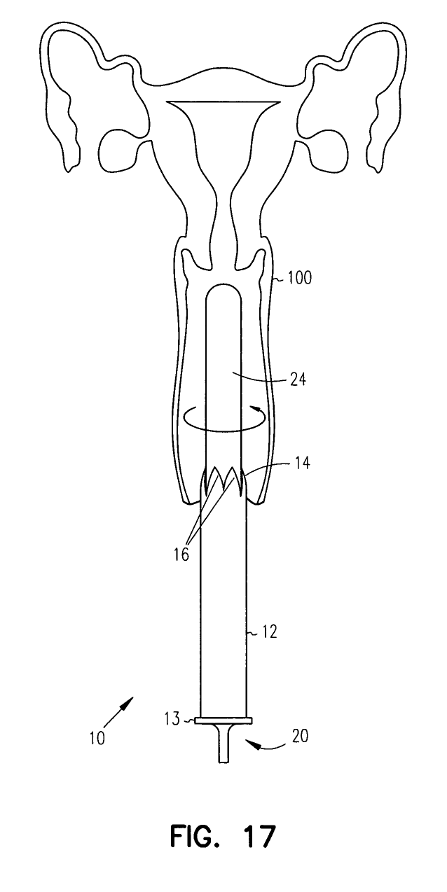

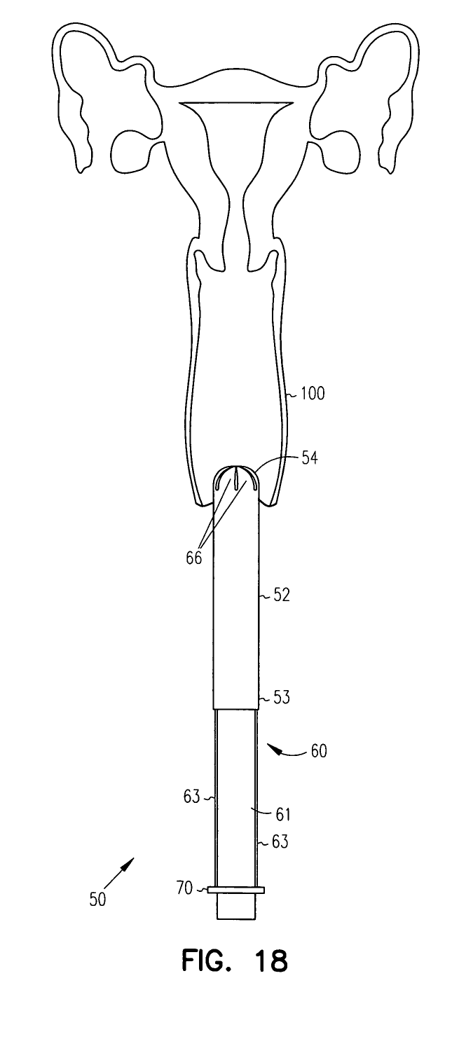

An example method of engaging a vagina will now be described with reference to FIGS. 16 and 17. The method includes inserting a disposable vaginal cleaning device 10 into a vagina 100. The disposable vaginal cleaning device 10 includes a housing 12 that has a first end 13 and a second end 14 and a core 20 that is positioned within the housing 12. The core 20 includes a handle 21 and a swab 24 (visible only in FIG. 17) that is secured to the handle 21. FIG. 16 shows that when the disposable device 10 is inserted into the vagina 100, the core 20 is in a first position where the swab 24 is at least partially within the housing 12 and a stop 23 on the handle 21 is outside the housing 12.

As shown in FIG. 17, the method further includes axially moving the core 20 to a second position where the swab 24 is extended through the second end 14 of the housing 12 further into the vagina 100 until the stop 23 on the handle 21 engages the housing 12 to prevent further insertion of the swab 24 into the vagina 100. In some embodiments, axially moving the core 20 to a second position may include forcing the swab 24 through resilient members 16 at the second end 14 of the housing 12; and/or (ii) engaging a stop 23 on the handle 21 with the first end 13 of the housing 12.

The method may further include rotating the handle 21 to clean inside the vagina 100 with the swab 24 and/or to apply a material (e.g., a lubricant) to the vagina with the swab 24. It should be noted that rotating the handle 21 may include engaging the handle 21 with the housing 12 to provide an indication as to how far the core 20 is rotating within the housing 12. In addition, engaging the handle 21 with the housing 12 may include engaging the handle 21 with the housing 12 when the core 20 is in the second position.

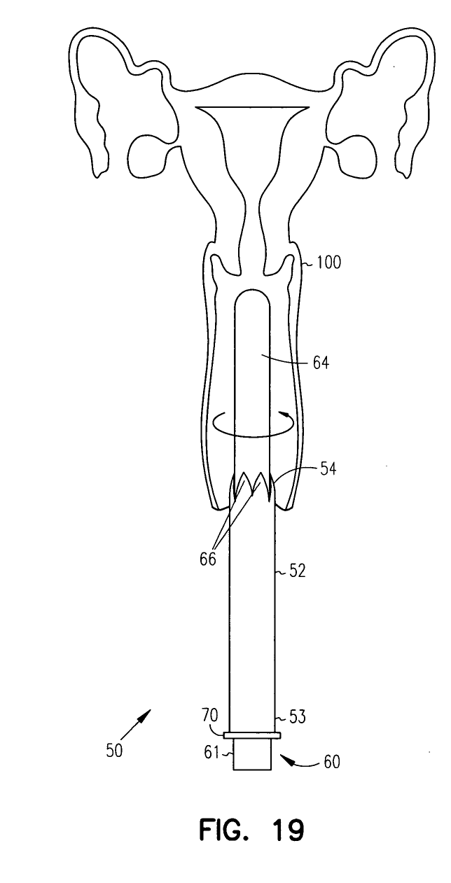

Another example method of engaging a vagina will now be described with reference to FIGS. 18 and 19. The method includes inserting a disposable vaginal insertion device 50 into a vagina 100. The disposable vaginal insertion device 50 includes a housing 52 having a first end 53 and a second end 54 and a core 60 that is positioned within the housing 52. The core 60 includes a handle 61 and a swab 64 (visible only in FIG. 17) that is secured to the handle 61. The method further includes rotating the handle 61 of the core 60 relative to the housing 52 to clean the vagina 100 with the swab 64 and to periodically engage the handle 61 with the housing 52. Periodically engaging the handle 61 with the housing 52 provides an indication to a user of the disposable device 10 how far the core 60 is rotating within the housing 52.

In some embodiments, inserting the disposable vaginal insertion device 50 into the vagina 100 may include inserting the disposable device 50 into the vagina 100 when the core 60 is in a first position where the swab 64 is at least partially within the housing 52 and a stop 70 on the handle 61 is outside the housing 52 (see FIG. 18). The method may further include axially moving the core 60 from the first position to a second position where the swab 64 is extended from the second end 54 of the housing 52 further into the vagina 100 until the stop 70 on the handle 61 engages the housing 52 (see FIG. 19). In the illustrated example embodiment, the handle 61 includes one or more members 63 (see FIG. 18) that periodically engage the housing 52 during rotation of the handle 61 when the core 60 is in the first position, the second position or anywhere in between.

Rotating the handle 61 of the core 60 to periodically engage the handle 61 with the housing 52 may include (i) generating a sound when the handle 61 periodically engages the housing 52; (ii) periodically applying a force that overcomes resistance generated by engaging the handle 61 with the housing 52; and/or (iii) engaging the handle 61 with the housing 52 one or more times per revolution of the core 60 relative to the housing 52.

Any of the disposable devices shown and/or described herein may used in any of the methods of engaging a vagina described herein. While the invention has been described in detail with respect to specific embodiments, it will be appreciated that there are variations of, and equivalents to these embodiments. Accordingly, the scope of the present invention should be determined by the appended claims and any equivalents thereto.

1. A disposable vaginal insertion device comprising:
a housing that includes a first end and a second end; and
a core positioned within the housing, the core including a handle and a swab that is secured to the handle, the core being axially movable within the housing between a first position where the swab is at least partially within the housing and a stop on the handle is outside the housing and a second position where the swab is extended through the second end of the housing and the stop on the handle engages the housing to prevent further insertion of the handle into the housing.

2. The disposable vaginal insertion device of claim 1 wherein the stop projects from the handle and engages the first end of the housing when the core is in the second position.

3. The disposable vaginal insertion device of claim 1 wherein the core moves within an opening in the housing such that the stop engages a projection on the housing that extends into the opening in the housing.

4. The disposable vaginal insertion device of claim 1 wherein the stop is an annular disk.

5. The disposable vaginal insertion device of claim 1 wherein the handle is embedded in the swab.

6. The disposable vaginal insertion device of claim 1 wherein the swab is positioned entirely within the housing when the core is in the first position.

7. The disposable vaginal insertion device of claim 6 wherein the swab is positioned entirely outside the housing when the core is in the second position.

8. The disposable vaginal insertion device of claim 6 wherein the second end of the housing includes resilient members that at least partially close the second end of the housing such that the resilient members are forced apart by the swab as the core moves from the first position to the second position.

9. The disposable vaginal insertion device of claim 1 wherein the core is able to rotate within the housing such that the handle periodically engages the housing to provide an indication as to how far the core is rotating within the housing.

10. The disposable vaginal insertion device of claim 9 wherein the handle periodically engages the housing when the core is in the second position.

11. A disposable vaginal insertion device comprising:
a housing that includes a first end and a second end; and
a core positioned within the housing, the core including a handle and a swab that is secured to the handle, the core being able to rotate within the housing such that the handle periodically engages the housing to provide an indication as to how far the core is rotating within the housing.

12. The disposable vaginal insertion device of claim 11 wherein the handle includes a first member that periodically engages the housing as the core rotates within the housing.

13. The disposable vaginal insertion device of claim 12 wherein the housing includes a detent that periodically receives the first member as the core rotates within the housing.

14. The disposable vaginal insertion device of claim 12 wherein the first member extends along the entire length of the handle.

15. The disposable vaginal insertion device of claim 11 wherein the core and the housing emit a sound when the handle periodically engages the housing.

16. The disposable vaginal insertion device of claim 11 wherein the handle engages the housing at least once per revolution of the core relative to the housing.

17. The disposable vaginal insertion device of claim 11 wherein the handle engages the housing at least twice per revolution of the core relative to the housing.

18. The disposable vaginal insertion device of claim 11 wherein the handle includes a first member on one side of the handle and a second member on an opposing side of the handle, the first and second members alternately engaging the housing as the core rotates relative to the housing.

19. The disposable vaginal insertion device of claim 11 wherein the housing includes a first projection on one side of the housing and a second projection on an opposing side of the housing, the first and second projections alternately engaging the handle as the core rotates relative to the housing.

20. The disposable vaginal insertion device of claim 11 wherein the housing includes a projection and the handle includes a detent that periodically receives the projection as the core rotates relative to the housing.

21. The disposable vaginal insertion device of claim 11 wherein the swab is embedded in the handle.

22. The disposable vaginal insertion device of claim 11 wherein the core is axially movable between a first position where the swab is at least partially within the housing and a stop on the handle is outside the housing, and a second position where the swab is extended through the second end of the housing until the stop engages the housing to prevent further insertion of the handle into the housing.

23. The disposable vaginal insertion device of claim 22 wherein the handle includes a first member that periodically engages the housing as the core rotates within the housing when the core is in the first position and the second position.

24. A method of engaging a vagina, the method comprising:
inserting a disposable vaginal insertion device into a vagina, the disposable vaginal insertion device including a housing having a first end and a second end and a core that is positioned within the housing, the core including a handle and a swab that is secured to the handle, the core being in a first position where the swab is at least partially within the housing and a stop on the handle is outside the housing; and
axially moving the core to a second position where the swab is extended through a second end of the housing further into the vagina until the stop on the handle engages the housing to prevent further insertion of the swab into the vagina.

25. The method of claim 24 further comprising rotating the handle to engage the vagina with the swab.

26. The method of claim 25 wherein rotating the handle includes engaging the handle with the housing to provide an indication as to how far the core is rotating within the housing.

27. The method of claim 26 wherein engaging the handle with the housing includes engaging the handle with the housing when the core is in the second position.

28. The method of claim 25 wherein rotating the handle to engage the vagina with the swab includes rotating the handle to clean inside the vagina.

29. The method of claim 25 wherein rotating the handle to engage the vagina with the swab includes rotating the handle to apply a material to the vagina.

30. The method of claim 24 wherein axially moving the core to a second position includes forcing the swab through resilient members at the second end of the housing.

31. The method of claim 24 wherein axially moving the core to a second position includes engaging the stop on the handle with the first end of the housing.

32. A method of engaging a vagina, the method comprising:
inserting a disposable vaginal insertion device into a vagina, the disposable vaginal insertion device including a housing having a first end and a second end and a core that is positioned within the housing, the core including a handle and a swab that is secured to the handle; and
rotating the handle relative to the housing to engage the vagina with the swab and to periodically engage the handle with the housing to provide an indication as to how far the core is rotating within the housing.

33. The method of claim 32 wherein inserting a disposable vaginal insertion device into a vagina includes inserting the disposable vaginal insertion device into the vagina when the core is in a first position where the swab is at least partially within the housing and a stop on the handle is outside the housing

34. The method of claim 33 further comprising axially moving the core to a second position where the swab is extended from the second end of the housing further into the vagina until the stop on the handle engages the housing to prevent further insertion of the swab into the vagina.

35. The method of claim 34 wherein rotating the handle to periodically engage the handle with the housing includes rotating the handle to periodically engage the stop on the handle with the housing when the core is in the second position.

36. The method of claim 32 wherein rotating the handle to periodically engage the handle with the housing includes generating a sound by periodically engaging the handle with the housing.

37. The method of claim 32 wherein rotating the handle to periodically engage the handle with the housing includes periodically applying a force that overcomes resistance generated by engaging the handle with the housing.

38. The method of claim 32 wherein rotating the handle to periodically engage the handle with the housing includes engaging the handle with the housing at least once per revolution of the core relative to the housing.

39. The method of claim 32 wherein rotating the handle to periodically engage the handle with the housing includes engaging the handle with the housing at least twice per revolution of the core relative to the housing.

40. The method of claim 32 wherein rotating the handle includes cleaning inside the vagina.

41. The method of claim 32 wherein rotating the handle includes applying a material to the vagina.
